(19) United States
(12) Patent Application Publication
(10) Pub. No.: US 2008/0242737 A1
(43) Pub. Date: Oct. 2, 2008

(54) Packaged oral delivery system containing a complexate

(76) Inventor: Richard Fuisz, McLean, VA (US)

Correspondence Address: HOFFMANN & BARON, LLP, 6900 JERICHO TURNPIKE, SYOSSET, NY, 11791, US

(21) Appl. No.: 11/731,381

(22) Filed: Mar. 30, 2007

(51) Int. Cl. A61K 47/04

(52) U.S. Cl. 514/770; 283/70; 434/367; 514/778; 514/789

(57) ABSTRACT

The present invention relates to packaged oral delivery systems for delivery of actives into the oral cavity. In particular, the packaged pharmaceutical product contains a complexate including a complexing agent and an active, an oral delivery system for delivery of the complexate and a package for containing the oral delivery system. The package includes indicia associated therewith. The indicia identifies the complexate as the active ingredient contained in the oral delivery system, which is as a regulatory approvable chemical entity. The present invention also relates to methods of labeling, pricing, marketing and satisfying governmental regulations for such packaged pharmaceutical products.

### FIELD OF THE INVENTION

The present invention relates to packaged oral delivery systems. The oral delivery systems contain new active ingredients for delivery into the oral cavity. The new active ingredient contained in the delivery system is a complexate and the package is labeled to indicate that such complexate is a regulatory approvable chemical entity.

### BACKGROUND OF THE INVENTION

Orally consumable pharmaceutical products contain active ingredients for treatment of a wide variety of ailments. The packaging and labeling of such products are heavily regulated to protect the safety of consumers. Specifically, pharmaceutical products are labeled to provide the consumer with accurate information as to the contents of the product. Regulatory bodies worldwide oversee safety in pharmaceutical product labeling. In the United States, the Food and Drug Administration (“FDA”) is responsible for regulating packaging and labeling of pharmaceutical products. Labeling for both prescription and over-the-counter (“OTC”) drug products are regulated by the FDA. In particular, regulations require accurate identification of all active ingredients and their amounts contained in the pharmaceutical product. For instance, for OTC products, actives must be listed in hierarchical order of their relative amounts along with the concentration per unit dosage and pharmaceutical purpose of each.

To comply with such regulations, active ingredient information must be correctly identified on the product labeling. Actives that are complexed with another substance, such as a salt, form a new chemical entity having new and different functionality from the initial uncomplexed active. This new chemical entity is the active ingredient that can be used in a variety of different pharmaceutical products. There is a need to properly identify this new chemical entity as the active ingredient contained in such products. Therefore, accurate drug information can be provided to consumers and governmental regulations can be satisfied.

### SUMMARY OF THE INVENTION

The present invention provides a new packaged oral delivery system which accurately provides information to a user regarding the active ingredient in the product. The active ingredients are new chemical entities that have previously not been recognized as such.

In accordance with some embodiments of the present invention, there is provided a packaged pharmaceutical product including a complexate including a complexing agent and an active, an oral delivery system for delivery of the complexate, and a package for containing the oral delivery system, the package including indicia associated therewith identifying the complexate as a regulatory approvable chemical entity.

Some embodiments provide a method of labeling a packaged pharmaceutical product in a manner that is consistent with governmental drug regulations, which includes the steps of:

(a) providing a packaged pharmaceutical product including:
(i) a complexate including a complexing agent and a pharmaceutical active;
(ii) an oral delivery system for delivery of the complexate; and
(iii) a package for containing the oral delivery system;

(b) adding indicia to the package, the indicia including a list of active ingredients contained in the oral delivery system and a list of inactive ingredients contained in the oral delivery system, wherein the complexate is listed as an active ingredient.

Some embodiments provide a method of adjusting the sales price of a packaged oral delivery system, which includes the steps of:

(a) selecting an oral delivery system including a pharmaceutical active, the product having a manufacturing cost;

(b) selecting a complexing agent for use with the pharmaceutical active to form a complexate;

(c) estimating the cost of purchasing the complexing agent from a supplier;

(d) estimating the cost of manufacturing the complexate;

(e) determining the change in manufacturing cost of the oral delivery system based on the additional cost estimates of steps (c) and (d); and

(f) adjusting the sales price of the product based on the change in manufacturing cost.

Some embodiments provide a method of disseminating accurate drug information to consumers of a packaged pharmaceutical product, which includes the steps of:

(a) providing a packaged pharmaceutical product including:
(i) a complexate including a complexing agent and a pharmaceutical active;
(ii) an oral delivery system for delivery of the complexate; and
(iii) a package for containing the oral delivery system;

(b) providing indicia identifying the complexate as an active ingredient contained in the packaged pharmaceutical product; and

(c) exposing consumers to the indicia, thereby providing accurate information as to the active ingredient contained in the packaged pharmaceutical product.

In some embodiments, there is a method of marketing an oral delivery system containing a complexate as the active ingredient to consumers, which includes the steps of:

(a) identifying a first pharmaceutical product containing an active that has been marketed to consumers as an effective pharmaceutical product;

(b) providing a second pharmaceutical product including:
(i) a complexate including a complexing agent and the active of step (a); and
(ii) an oral delivery system for delivery of the complexate;

(c) educating consumers that the complexate contained in the second pharmaceutical product provides the same effectiveness as the active contained in the first pharmaceutical product; and

(d) marketing the second pharmaceutical product to consumers.

Some embodiments provide a method of developing a sales price for a packaged oral delivery system including a complexate as the active ingredient, including the steps of:

(a) selecting a pharmaceutical active and complexing agent to form the complexate included in the oral delivery system;

(b) estimating the cost of purchasing the complexing agent from a supplier;

(c) estimating the cost of purchasing the pharmaceutical active from a supplier;

(d) estimating the cost of manufacturing the complexate from the pharmaceutical active and the complexing agent;

(e) estimating the cost of producing and packaging the oral delivery system including the complexate;

(f) estimating the aggregate cost of safety and efficacy tests performed for the pharmaceutical active and the complexing agent individually;

(g) determining whether additional safety and efficacy tests for the complexate are required;

(h) estimating the cost associated with the additional safety and efficacy tests of step (g);

(i) adding the costs of the preceding steps to develop an estimated total manufacturing cost; and

(j) setting the sales price based on the estimated total manufacturing cost of the packaged oral delivery system.

Still other embodiments provide a method of satisfying drug regulations promulgated by a regulatory body for an oral delivery system including a complexate as the active ingredient, the complexate including a complexing agent and a pharmaceutical active, which includes the steps of:

(a) submitting safety and efficacy test results for the complexing agent and safety and efficacy test results for the pharmaceutical active to the regulatory body;

(b) determining whether additional safety and efficacy tests for the complexate are required by the regulatory body;

(c) conducting the safety and efficacy tests of step (b); and

(d) submitting the results of the safety and efficacy tests of step (c) to the regulatory body to satisfy the drug regulations of the regulatory body for the oral delivery system.

### BRIEF DESCRIPTION OF THE DRAWINGS

FIG. 1 is a side elevation view of a packaged pharmaceutical product in accordance with some embodiments of the present invention.

FIG. 2 is a side elevation view of a packaged pharmaceutical product including a blister pack in accordance with some embodiments of the present invention.

### DETAILED DESCRIPTION OF THE INVENTION

The present invention relates to packaged pharmaceutical products that can be commercially marketed and sold to consumers. The packaged pharmaceutical products appropriately identify the active ingredient contained therein as an active that has obtained regulatory approval as a new chemical entity. More specifically, the packaged pharmaceutical products are oral delivery systems. The oral delivery systems include an active ingredient for delivery into the oral cavity. The active ingredient contained in the oral delivery systems is a complexate, which has new and different functionality over the initial uncomplexed active, as well as new clinical advantages.

A “complexate” is a chemical entity that is formed by a chemical complex, association or interaction between an active, such as a charged active, and a complexing agent, such as an acidic or basic resin. The resulting material formed from these two components is referred to herein as a “complexate”. The complexate is a distinct chemical entity over the initial uncomplexed active. This chemical entity exhibits different functionality and offers new clinical advantages over the initial active. Furthermore, the complexate is a new chemical entity for purposes of FDA regulatory approval. For instance, if the complexate is considered a new chemical entity by the FDA, regulatory approval for use of the complexate as an active ingredient in oral delivery systems would be required by the FDA. The package of the oral delivery system is labeled to reflect that this complexate is one of the active ingredients contained in the product, rather than merely listing the initial uncomplexed active as the active ingredient. Therefore, the product packaging provides accurate disclosure information to consumers as to the active ingredient contained in the product.

The package containing the oral delivery system may be any conventional packaging material used for pharmaceutical products. For example, the package may be, without limitation, a pouch, box, cassette, blister pack, bag, bottle, syringe, vial, tube, or the like.

The package containing the oral delivery system bears indicia thereon. The indicia provides information about the product ingredients to the consumer. In the pharmaceutical context, this information is extremely important to consumers who select products based on the active ingredients contained therein. Moreover, as discussed above, the listing of actives contained in pharmaceutical products is regulated by the FDA. The indicia on the package includes a list of active ingredients contained in the oral delivery system. The indicia identifies the complexate included in the oral delivery system as an active ingredient contained therein. Because the complexate is a regulatory approvable chemical entity for use in pharmaceutical products, it is appropriately identified as an active ingredient on the product indicia. Additional indicia also may include a list of inactive ingredients contained in the oral delivery system.

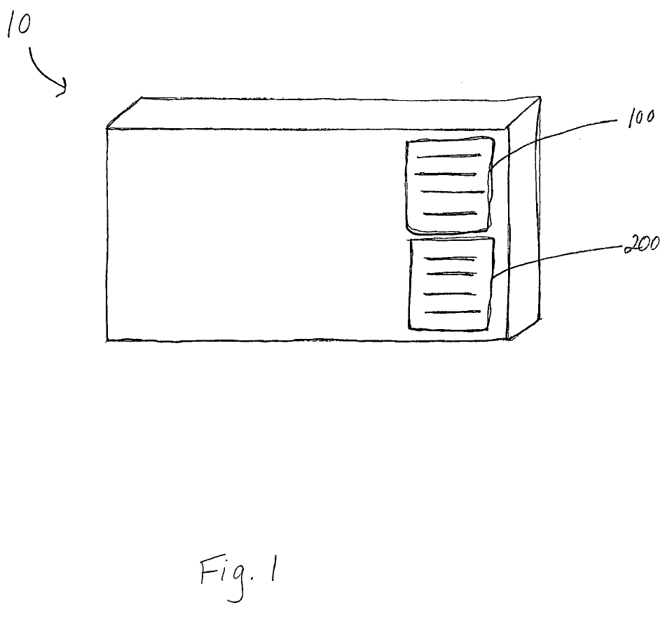

The indicia included on the packaged product may take a wide variation of forms, such as labels, symbols, bar codes, patterns and other means of communicating information. One preferred indicia is a label. The label or other indicia may be located on or affixed to any packaging component or the product per se. Any conventional means for affixing labels to a product package may be employed. For instance, as shown in FIG. 1, the packaged pharmaceutical product 10 may include multiple sets of indicia 100 and 200. The indicia of each set may be different. For example, the first indicia 100 may include a listing of active ingredients included in the oral delivery system contained in the package 10. The second indicia 200 may include a listing of inactive ingredients included in the oral delivery system contained in the package 10. The oral delivery system housed inside the package 10 includes a complexate. As mentioned above, the complexate is a chemical entity formed from a complexing agent and an active. Accordingly, the complexate itself is the active ingredient contained in the oral delivery system. The complexate as a chemical entity itself is listed in the indicia 100 as an active ingredient contained in the oral delivery system. Therefore, the indicia appropriately conveys to the consumer that the complexate is one of the active ingredients contained in the oral delivery system.

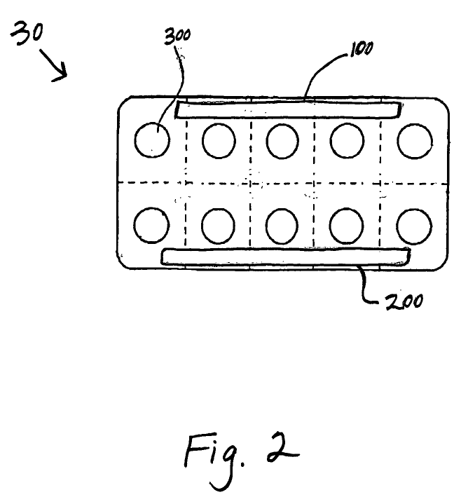

In another embodiment shown in FIG. 2, for example, a plurality of oral delivery systems may be housed inside a blister pack. For example, as shown in FIG. 2, a blister pack 30 may contain a plurality of tablets 300. The tablets 300 include a complexate as an active ingredient. The blister pack 30 may include two different sets of indicia 100 and 200 on the outer surface thereof. As described above, indicia 100 includes a listing of active ingredients included in the tablets 300. Indicia 200 includes a listing of inactive ingredients included in the tablets 300. The complexate included in the tablets 300 is included in the list of active ingredients in indicia 100. The indicia 100 and 200 appropriately indicate to consumers the active and inactive ingredients contained in the product.

It should be understood that any type of packaging may be used to house the oral delivery systems and convey the information regarding the identification of the complexate as an active ingredient. Suitable oral delivery systems and complexates for use in the packaged pharmaceutical products will be described in more detail below.

### Oral Delivery Systems

As described above, the packaged pharmaceutical products described herein include an oral delivery system for delivery of an active ingredient. The oral delivery systems dissolve or disintegrate in the oral cavity and particularly suitable for delivery of actives. The active ingredient delivered by the oral delivery systems described herein is a complexate. A variety of different oral delivery systems typically used in pharmaceutical products may be employed, such as, but not limited to, tablets, capsules, caplets, lozenges, liquids, flash dose, sachets, aerogels, nanoparticles and granules, among others. Exemplary delivery systems are described in more detail below.

### Tablets

Tablets are conventionally formed by compressing a granulated formulation of the components together to form a tablet. Tablet formulations typically include binders, flow agents, lubricants, glidants, fillers, disintegrants, surfactants, fillers/compression aids, solvents and the like.

Examples of binders include, but are not limited to, hydroxypropyl methylcellulose, PVP, hydroxypropyl cellulose, microcrystalline cellulose, hydroxymethylcellulose, carbopol and sodium carboxymethylcellulose, acacia, alginic acid, carbomer, dextrin, ethylcellulose, gelatin, guar gum, hydrogenated vegetable oil (type I), hydroxyethyl cellulose, hydroxypropyl methylcellulose, liquid glucose, magnesium aluminaum silicate, maltodextrin, methylcellulose, polymethacrylates, povidone, pregelatinized starch, sodium alginate, starch, and zein.

Examples of disintegrants include, but are not limited to, alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polyacrilin potassium, powdered cellulose, pregelatinized starch, sodium alginate and starch.

Examples of fillers (also referred to as a diluents) include, but are not limited to, calcium carbonate, calcium sulfate, compressible sugars, confectioner's sugar, dextrates, dextrin, dextrose, dibasic calcium phosphate dihydrate, glyceryl palmitostearate, hydrogenated vegetable oil (type I), kaolin, lactose, magnesium carbonate, magnesium oxide, maltodextrin, mannitol, polymethacrylates, potassium chloride, powdered cellulose, pregelatinized starch, sodium chloride, sorbitol, starch, sucrose, sugar spheres, talc and tribasic calcium phosphate.

Examples of fillers/compression aids include, but are not limited to, lactose, calcium carbonate, calcium sulfate, compressible sugars, dextrates, dextrin, dextrose, calcium phosphate, kaolin, magnesium carbonate, magnesium oxide, maltodextrin mannitol, powdered cellulose, pregelatinized starch, and sucrose.

Examples of surfactants include, but are not limited to, anionic and cationic surfactants, such as sodium lauryl sulfate, docusate sodium (dioctyl sulfosuccinate sodium salt), benzalkonium chloride, benzethonium chloride, and cetrimide (alkyltrimethylammonium bromide, predominantly C.sub.14 alkyl).

Examples of lubricants include, but are not limited to, calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate.

Examples of suitable solvents include, but are not limited to, water, ethanol or mixtures thereof.

Tablets typically are formed by wet granulation, dry granulation or compression methods. In wet granulation processes, a tablet may be formed by blending the components together, such as a filler/disintegrant, binder, surfactant and the complexate, adding water to wet granulate the blended mixture to agglomerate the mixture, drying the granulated mixture, milling the dried mixture to granulate to a uniform size, blending the milled mixture with a compression aid, lubricating the blended mixture with a lubricant and compressing the lubricated mixture to a compressed tablet of the desired shape.

As mentioned above, granulation also may be achieved by conducting dry granulation (without water) using a roller compaction process. In dry granulation processes, a powder mixture is granulated by compression without the use of heat and solvent. Two methods typically are used. One method is slugging, where the powder is precompressed on a heavy-duty tablet press, and the resulting tablets or slugs are milled to yield the granulation. The other method is precompression of the powder with pressure rolls using a compactor.

Direct compression tabletting involves compressing tablets directly from powder blends of the active ingredient, i.e., the complexate, and suitable excipients (including fillers, disintegrants and lubricants) which are included in the mix to provide uniform flow into the die cavity and form a firm solid compression tablet. No pretreatment of the powder blends by wet or dry granulation procedures is needed.

The resulting tablet then may be film coated with a film coating suspension to produce a film coated tablet. Film coating suspensions typically include one or more of the following components: carboxymethylcellulose sodium, carnauba wax, cellulose acetate phthalate, cetyl alcohol, confectioner's sugar, ethyl cellulose, gelatin, hydroxyethyl cellulose, hydroxypropyl cellulose, hydroxypropyl methylcellulose, liquid glucose, maltodextrin, methyl cellulose, microcrystalline wax, Opadry and Opadry II, polymethacrylates, polyvinyl alcohol, shellac, sucrose, talc, titanium dioxide, and zein.

### Capsules

Capsules generally are elongated, cylindrical dosage forms. Typically, capsules are made of gelatin, starch, methyl cellulose, sugar-gelatin or gelatin-glycerin. Capsules generally include two separate parts, i.e., two semicapsules, one a cap and the other a body. The capsule may be filled with a pharmaceutical composition, generally a powder or liquid including the complexate, and the capsule dosage form completed by placing the capsule cap over the capsule body.

Soft gelatin capsules or softgels are predominantly used to contain liquids wherein the active ingredients are present in the dissolved or suspended state. Filled one-piece softgels have been widely known and used for many years and for a variety of purposes. Because softgels have properties that are quite different from telescoping two-piece, hard shell capsules, the softgels are capable of retaining a liquid fill material. Softgels commonly are used to enclose consumable materials such as vitamins and pharmaceuticals in a liquid vehicle or carrier.

In some embodiments, the capsule cap may be prepared by combining appropriate amounts of gelatin, water, plasticizer, and any optional components in a suitable vessel and agitating and/or stirring while heating to about 65° C. until a uniform solution is obtained. This cap can then be used for encapsulating the capsule body, containing the desired quantity of the solubilized fill composition, employing standard encapsulation methodology to produce one-piece, hermetically-sealed, soft gelatin capsules.

Acceptable gelatin compositions are any of those known to one of ordinary skill in the art and can contain small amounts of methyl cellulose, polyvinyl alcohols, and denatured gelatins to modify their solubility or produce a enteric effect. Common sources of gelatin contemplated by this invention include animal bones, the skin of cold water fish, hide portions and frozen pork skin. Grades of gelatin that are appropriate for this invention include pharmaceutical grade, food grade, Type A and Type B, and combinations thereof. One example of a gelatin suitable for capsule manufacture may be such as those commercially available from the Sigma Chemical Company, St. Louis, Mo. For a general description of gelatin and gelatin-based capsules, see Remington's Pharmaceutical Sciences, 16th ed., Mack Publishing Company, Easton, Pa. (1980), page 1245 and pages 1576-1582; and U.S. Pat. No. 4,935,243, to Borkan et al., issued Jun. 19, 1990; these two references being incorporated herein by reference in their entirety.

Additionally, plasticizers may be incorporated to produce a soft gelatin shell. The soft gelatin thus obtained has the required flexibility characteristics for use as an encapsulation agent. Useful plasticizers include glycerin, sorbitan, sorbitol, or similar low molecular weight polyols, and mixtures thereof.

### Caplets

Caplets are generally capsule shaped tablets, or elongated solid member formed from a pharmaceutical composition. Caplet formulations typically include binders, flow agents, lubricants, fillers, disintegrants, surfactants, fillers/compression aids, solvents and the like. Suitable materials include those which will absorb water and swell. The most common materials of this nature are cellulose derivatives, although high molecular weight proteins and synthetic polymers are also employed. Typically useful materials which can be utilized include methyl cellulose, hydroxypropyl methylcellulose available as “Methocel” (available from Dow Chemical, Midland, Mich.); hydroxypropyl cellulose, available as “Klucel” from Aqualon Co; Povidone; sodium carboxymethyl cellulose; carboxymethyl cellulose; “Carbopol” water soluble gel forming polyacrylic resin available form B.F. Goodrich Chemical Co. and sorbitol. Additional examples of acceptable binders, flow agents, lubricants, fillers, disintegrants, surfactants, fillers/compression aids, solvents include any of those which are discussed in detail above.

A caplet may be made by blending the components together, such as a filler/disintegrant, binder, surfactant and the complexate, blending the milled mixture with a compression aid, lubricating the blended mixture with a lubricant and compressing the lubricated mixture to a compressed tablet of the desired shape. In some embodiments, caplets may be formed in a conventional automatic tableting machine. The caplet is generally formed by tableting machines by applying pressure on the sides of the caplet, which results in the formation of a ring-like protrusion about the longitudinal sides of the caplet.

In some embodiments, formed caplets may then be coated with a water-soluble film coating to provide a barrier for those active material agents that may adversely interact with the capsule material.

### Flash Dose Units

Rapidly dissolving, tablet-like dosage forms also may be prepared by flash flow processes to provide a shearform matrix that is subsequently compressed to form comestible compression units. Such dosage units dissolve nearly instantaneously in the oral cavity of the consumer.

In accordance therewith, an uncured shearform matrix and an additive, such as the complexate, are mixed together to prepare for molding a unit dosage. A shearform matrix is a matrix that is produced by subjecting a feedstock that contains a carrier material to flash flow processing.

Flash flow processing can be accomplished several ways. Flash-heat and flash-shear are two processes which can be used. In the flash-heat process the feedstock material is heated sufficiently to create an internal flow condition which permits part of the feedstock to move at subparticle level with respect to the rest of the mass and exit openings provided in the perimeter of a spinning head. The centrifugal force created in the spinning head flings the flowing feedstock material outwardly from the head so that it reforms with a changed structure. The force necessary to separate and discharge flowable feedstock is centrifugal force which is produced by the spinning head.

One preferred apparatus for implementing a flash heat process is a “cotton candy” fabricating type of machine. The spinning machine used to achieve a flash-heat condition is a cotton candy machine such as the Econo-Floss Model 3017 manufactured by Gold Medal Products Company of Cincinnati, Ohio. Any other apparatus or physical process which provides similar forces and temperature gradient conditions can also be used.

In the flash-shear process, a shearform matrix is formed by raising the temperature in the feedstock material which includes a non-solubilized carrier, such as a saccharide-based material until the carrier undergoes internal flow upon application of a fluid shear force. The feedstock is advanced and ejected while in internal flow condition, and subjected to disruptive fluid shear force to form multiple parts or masses which have a morphology different from that of the original feedstock.

The multiple masses are cooled substantially immediately after contact with the fluid shear force and are permitted to continue in a free-flow condition until solidified.

The flash shear process can be carried out in an apparatus which has means for increasing the temperature of a non-solubilized feedstock and means for simultaneously advancing it for ejection. A multiple heating zone twin screw extruder can be used for increasing the temperature of the non-solubilized feedstock. A second element of the apparatus is an ejector which provides the feedstock in a condition for shearing. The ejector is in fluid communication with the means for increasing the temperature and is arranged at a point to receive the feedstock while it is in internal flow condition. The ejector is preferably a nozzle which provides high pressure ejection of the feedstock material. See U.S. Pat. No. 5,380,473, filed Oct. 23, 1992 entitled “Process For Making Shearform Matrix,” which is incorporated herein by reference.

The feedstock for producing shearform matrix includes a carrier material. The carrier material can be selected from material which is capable of undergoing both physical and/or chemical changes associated with flash-flow processing. Materials useful as matrices may be chosen from those carbohydrates which are capable of forming free-form agglomerates upon being processed.

Suitable materials useful as matrices may be chosen from such classes as “sugars”. “Sugars” are those substances which are based on simple crystalline mono- and di-saccharide structures, i.e., based on C₅ and C₆ sugar structures. “Sugars” include sucrose, fructose, lactose, maltose, and sugar alcohols such as sorbitol, mannitol, maltitol, and the like. Other carrier materials may be used in combination with the sugars, such as maltodextrins, maltooligosaccharides, polydextrose, and the like. Optional additives, such as flavors and sweeteners, also may be used.

A shearform product is used to obtain the new sugar product. A shearform sugar product is a substantially amorphous sugar which results from subjecting sugar to heat and shear sufficient to transform crystalline (usually granulated) sugar to amorphous sugar without the use of a solution. Thus, a shearform sugar product is characterized as a sugar product resulting from a non-solubilized sugar.

The shearform matrix is initially uncured, which enables formation of a dosage unit upon curing. An additive, e.g., the complexate, is mixed with the uncured shearform matrix. The mixture then is molded as a unit dosage form. More specifically, the uncured (i.e., uncrystallized) shearform matrix material is associated closely enough to provide bridging between crystallized matrix material upon curing. Generally, this requires force sufficient to provide intimate contact of fibers prior to curing, followed by crystallizing to form a bound continuous crystalline structure throughout the tablet. Unlike conventional tabletting which relies primarily on compression to provide the structure, this process utilizes the curing process to aid in forming the end product. Consequently, mild compression forces can be used to mold the product. In a preferred embodiment, the compression required to mold uncured matrix material is referred to as “tamping.”

“Tamping” means compressing with force less than that required in compression tabletting, which is generally regarding as being on the order of thousands of pounds per square inch (psi). The maximum pressure used is only 500 psi, but in most cases will never exceed about 250 psi, and, in the most preferred embodiments, not more than 80 psi (e.g., 40 psi to 80 psi). These lower pressures are called tamping.

After preparing shearform matrix and molding the uncured matrix, the product is cured. Curing means binding and crystallizing the matrix material substantially simultaneously. Curing is performed by subjecting product to heat and moisture sufficient to provide controlled crystallization. Controlled crystallization occurs when points of contact of uncured matrix material become points of crystalline growth and crystallization of the material proceeds to provide crystalline structures. Binding occurs at the points of contact, and the simultaneous crystalline growth is such as to maintain structural integrity. The curing process involves a transformation from amorphous to crystalline state. The transformation takes place while the amorphous shearform matrix remains bound together.

For a more detailed discussion of such dosage units, see U.S. Pat. Nos. 5,622,719, 5,851,553, 5,866,163 and 5,871,781, the contents each of which are incorporated herein by reference.

### Liquid Compositions

Liquid compositions include a variety of compositions, such as syrups, elixirs, suspensions, sprays, and the like. For liquid compositions, the carrier desirably includes some of the following optional ingredients: water; sweetening agents, such as sucrose, corn syrup, invert sugar, dextrose, sodium saccharin, aspartame, sorbitol, honey, and magnasweet; aromatic ingredients, such as menthol, anethol, camphor, thymol, methyl salicylate, eucalyptus oil and peppermint oil; other flavoring agents; thickening agents, such as carboxymethylcellulose, sodium carboxymethylcellulose, cellulose, glycerine and polyethylene glycol; coloring agents; preservatives, such as sodium benzoate and cetylpyridinium chloride; miscellaneous ingredients, such as potassium sorbate, sodium chloride, titanium dioxide, polysorbate 80, sodium citrate, sodium bicarbonate, sodium hydroxide, aluminum hydroxide and magnesium hydroxide.

### Complexates

In accordance with the present invention, the active ingredient included in the oral delivery system is a complexate, which includes a complexing agent and an active. The complexate is a different chemical entity from the initial uncomplexed active. In some embodiments, for instance, the initial active is a charged active, which allows it to complex, interact or associate with an oppositely charged complexing agent, the formation of which is identifiable as a new active ingredient, i.e., a complexate.

The complexing agent may be any agent capable of chemically forming a complex, association or interaction with an active. For instance, the complexing agent may be an anionic complexing agent, which may react with an active having a cationic charge to form a complexate. Alternatively, the complexing agent may be a cationic complexing agent, which may react with an active having an anionic charge to form a complexate. In some embodiments, the complexing agent may be a multi-valent salt, which also may react with a charged active to form a complexate. In some embodiments, the complexing agent may be a zwitterion, which is a molecule carrying both a positive and a negative charge, and thus, can form a complex with a charged active. In some embodiments, the complexing agent may be an adsorbant or an absorbant material, such as, trimagnesium silicate or other absorbing silicates.

More specifically, in some embodiments, the complexing agent may be an ion exchange resin. Ion exchange resins may serve several different functions in pharmaceutical applications, including extended- or controlled-release, taste-masking, and improving the stability of actives. Ion exchange resins generally are insoluble macromolecules or polyelectrolytes that have electrically charged sites at which one ion may replace another ion. Cation-exchange resins have fixed electronegative charges that interact with counterions having the opposite, or positive, charge. Cation-exchange resins exchange positively charged cations. Anion-exchange resins have electropositive charges that interact with counterions having the opposite, or negative, charge. Anion-exchange resins exchange negatively charged anions.

In particular, an ion exchange resin for use herein may be a water-insoluble organic or inorganic matrix material having covalently bound functional groups that are ionic or capable of being ionized under appropriate conditions. The organic matrix may be synthetic (e.g., polymers or copolymers or acrylic acid, methacrylic acid, sulfonated styrene or sulfonated divinylbenzene) or partially synthetic (e.g., modified cellulose or dextrans). The inorganic matrix may be, for example, silica gel modified by the addition of ionic groups. Most ion exchange resins are cross-linked by a crosslinking agent, such as divinylbenzene.

Ion exchange resins for use herein may be categorized into four main types depending on their functional groups: strongly acidic (e.g., sulfonic acid groups); strongly basic (e.g., trimethylammonium groups); weakly acidic (e.g., carboxylic acid groups); and weakly basic (e.g., amino groups).

In some embodiments, for instance, an acidic resin may be employed. The acidic resin may be combined with a basic drug to form a complexate. Examples of acidic resins that can be combined with basic drugs include, but are not limited to, partially neutralized poly(acrylic acid), crosslinked acrylic acid copolymers (such as Indion 414), sodium polystyrene sulfonate (such as Amberlite IRP-69), copolymers of methyacrylic acid crosslinked with divinylbenzene (such as Amberlite IRP-64), and polacrilin potassium.

Examples of basic drugs that can be combined with any of the acidic resins set forth above include, but are not limited to, levobetaxolol hyrdrochloride, roxithromycin, dicyclomine hydrochloride, montelukast sodium, dextromethorphan hydrobromide, diphenhydramine hydrochloride, orbifloxacin, ciprofloxacin, enoxacin, grepafloxacin, levofloxacin, lomefloxacin, nalidixic acid, acycloguanosine, tinidazole, deferiprone, cimetidine, oxycodone, remacemide, nicotine, morphine, hydrocodone, rivastigmine, propanolol, betaxolol, chlorpheniramine, and paroxetine.

In some embodiments, a basic resin may be employed. The basic resin may be combined with an acidic drug to form a complexate. Examples of basic resins that can be used to form complexates include, but are not limited to, polyvinylpyrrolidone, polylysine, polyarginine, and polyhistidine.

Examples of acidic drugs that can be combined with any of the basic resins set forth above include, but are not limited to, nicotinic acid, mefanamic acid, indomethacin, diclofenac, repaglinide, ketoprofen, ibuprofen, valproic acid, lansoprazole, ambroxol, omeprazole, acetaminophen, topiramate, amphotericin B, and carbemazepime.

In some other embodiments, the complexing agent may rely on weak bonding forces, such as Van der Waals forces or hydrogen bonding, to form a complexate with an initial active. Such complexing agents may include caged molecules, such as cyclodextrins. Cyclodextrins generally are cyclic oligosaccharides composed of alpha-D-glucopyranose units. Common cyclodextrins include alpha-, beta- and gamma-cyclodextrins, which contain 6, 7 and 8 glucose units, respectively. Cyclodextrins have a toroidal shape with a generally hydrophobic interior cavity and a generally hydrophilic exterior, which imparts water-solubility to the molecule. This characteristic allows cyclodextrins to form inclusion complexes, i.e., host-guest complexes, with hydrophobic molecules to increase the water-solubility thereof. More specifically, guest molecules interact with the interior cavity of the cyclodextrin to become entrapped and form a stable association therewith. Due to the hydrophilic exterior of the cyclodextrin, the inclusion complex is water-soluble, thereby increasing the release of poorly soluble drugs complexed therewith.

Examples of such complexing agents include, but are not limited to, alpha-cyclodextrin, beta-cyclodextrin, gamma-cyclodextrin and derivatives of cyclodextrins, such as hydroxyalkylated cyclodextrins. Examples of drugs that can be combined with this type of complexing agent are many and are determined by the fit of the drug within the complexing agent, e.g., cyclodextrin. For example, anthracyclines form good complexes with gamma-cyclodextrin. Complexes of other cyclodextrins are described in U.S. Pat. No. 4,727,064, which is incorporated herein by reference in its entirety.

In some embodiments described herein, the complexing agent may be a zeolite. Zeolites are minerals having a micro-porous structure. Zeolites include naturally occurring minerals and synthetic compounds, which generally are characterized by an alumino-silicate framework with an open structure that can accommodate cations, such as Na⁺, K⁺, Ca²⁺, Sr²⁺ and Ba²⁺. The cations reside in cavities in the crystal structure and can be readily exchanged for others in a solution. Zeolites can be of various different types, such as P-type and X-type, and with numerous counterions, such as sodium and calcium. Additionally, zeolites can be used in combination with ammonium salts, such as hexadecyltrimethyl ammonium bromide. An example of this is a complex of chloroquin with a P-type zeolite with a sodium counterion and in the presence of dodecyltrimethylammonium bromide.

In some embodiments, the complexing agent may rely on any type of molecular entanglement, as such entanglement is understood in quantum theory. Any materials that are bound in any way are by definition “entangled” in quantum theory.

In such embodiments, the molecular chains of a complexing agent, such as a polymer, are sufficiently entangled to trap or bind the active, thereby forming the complexate. In instances when the molecular weight is excessive, the ability of the thus formed complexate to release the active may be hampered or too slow for practical purposes. Thus, the upper limit for molecular weight of the complexing agent is that which still provides efficacy for its intended use. The upper limits of molecular weight will of course depend on the polymer chosen, as well as the active, since the behavior of the complexate is dependent to a large degree on its formative components.

In some embodiments, the complexing agent may promote volatilization.

In addition to the drugs specifically provided above, any of a variety of pharmaceutical actives, medicaments and bioactive active substances may be used in forming the complexates. The following is a non-exhaustive list of exemplary actives.

A wide variety of medicaments, bioactive active substances and pharmaceutical actives may be employed. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of medicating active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine.

Other preferred drugs for other preferred active ingredients for use in the present invention include anti-diarrheals such as immodium AD, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the oral delivery systems of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™), nabumetone (available as Relafen®), and Clemastine (available as Tavist®); anti-emetics such as granisetron hydrochloride (available as Kytril®) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®).

Erectile dysfunction therapies include, but are not limited to, drugs for facilitating blood flow to the penis, and for effecting autonomic nervous activities, such as increasing parasympathetic (cholinergic) and decreasing sympathetic (adrenersic) activities. Useful non-limiting drugs include sildenafils, such as Viagra®, tadalafils, such as Cialis®, vardenafils, apomorphines, such as Uprima®, yohimbine hydrochlorides such as Aphrodyne®, and alprostadils such as Caverject®.

The popular H₂-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents employed in the present invention may include allergens or antigens, such as, but not limited to, plant pollens from grasses, trees, or ragweed; animal danders, which are tiny scales shed from the skin and hair of cats and other furred animals; insects, such as house dust mites, bees, and wasps; and drugs, such as penicillin.

An anti-oxidant may also be added to the oral delivery system to prevent the degradation of an active, especially where the active is photosensitive.

Cosmetic active agents may include breath freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like.

In addition, in some embodiments, the complexate may be taste-masked prior to incorporation into the oral delivery system.

In any of the embodiments described above, the active may be present in amounts of about 0.01% to about 60% by weight of the complexate. The complexing agent may be present in amounts of about 0.2% to about 20% by weight of the complexate. The complexing agents described above may have molecular weights of about 2,000 or more.

### Methods of Labeling, Pricings, Marketing and Satisfying Regulations for Complexates

The present invention also is directed to methods of labeling a packaged pharmaceutical product in a manner that is consistent with governmental drug regulations. More specifically, the FDA regulates the packaging requirements for pharmaceutical products in the United States. Product packaging must include a listing of the active ingredients contained in the product. Inactive ingredients are listed separately. In accordance with some embodiments described herein, a packaged pharmaceutical product is provided. The packaged pharmaceutical product includes a complexate, as described above, an oral delivery system for delivery of the complexate and a package for containing the oral delivery system. Indicia are added to the package, which include a list of active ingredients contained in the oral delivery system and a list of inactive ingredients contained in the oral delivery system. The complexate is included in the list of active ingredients on the product package. In particular, as discussed above, the complexate is the active ingredient itself due to its interaction with an initial active. Once the complexing agent interacts with the active to form the complexate, the initial active no longer exists. Therefore, listing the initial active on the product packaging would not be consistent with FDA regulations. Rather, the active ingredient contained in the oral delivery system is the complexate. Therefore, including the complexate in the list of active ingredients on the indicia is consistent with FDA regulations. Moreover, listing the complexate as such appropriately informs consumers as to the active ingredient contained in the product.

The present invention also provides methods of adjusting the sales price of a packaged oral delivery system. In accordance with such methods, an oral delivery system, which includes a pharmaceutical active, is selected. The manufacturing cost of the oral delivery system is known. A complexing agent then is selected for use with the pharmaceutical active. Once the particular complexing agent is selected, the cost of purchasing the complexing agent from a supplier is estimated. Additionally, the cost of manufacturing the complexate from the selected complexing agent and the pharmaceutical active is estimated. Subsequently, a determination is made as to the change in manufacturing cost of the oral delivery system based on these additional cost estimates. Based on this change in manufacturing cost, the sales price of the product may be adjusted. For instance, the cost of purchasing the complexing agent may add a substantial increase to the cost of manufacturing the product. As such, the sales price per unit of the product may be adjusted upwardly to account for the increased cost.

The sales price of the oral delivery system also may be adjusted to compensate for various other costs. For instance, the modification of the product to include a complexate rather than the original active may require additional safety and efficacy tests. There are costs associated with conducting such clinical tests, as well as submitting the results of the tests to the requisite regulatory body for approval. The modification of the product to include a complexate also may require a labeling change on the product packaging. The development and production of new packaging also involves increased costs. The sales price per unit of the product may be adjusted based on these additional costs, as well.

Finally, a determination may be made as to whether or not the adjusted sales price of the product is viable in the consumer marketplace. If the adjusted sales price is not viable, all of the manufacturing costs may need to be reexamined to determine where costs can be reduced.

Some embodiments of the present invention are directed to methods of developing a sales price for a packaged oral delivery system including a complexate as the active ingredient. A pharmaceutical active and complexing agent are selected for forming the complexate to be included in the oral delivery system. The cost of purchasing the complexing agent from a supplier is estimated, as well as the cost of purchasing the pharmaceutical active from a supplier. The cost of manufacturing the complexate from the pharmaceutical active and the complexing agent also is estimated, as well as the cost of producing and packaging the oral delivery system including the complexate. The aggregate cost of safety and efficacy tests performed for the pharmaceutical active and the complexing agent individually is estimated. Then, a determination is made as to whether or not additional safety and efficacy tests for the complexate are required. Additional safety and efficacy tests may be required by a regulatory body, e.g., the FDA, to satisfy governmental drug regulations. If such additional tests are required, the costs associated with conducting such tests and submitting the results to the regulatory body are estimated. Finally, all of the cost estimates are added together to develop an estimated total manufacturing cost for the product and then the sales price may be set based on this estimated total manufacturing cost.

The present invention additionally is directed to methods of disseminating accurate drug information to consumers of a packaged pharmaceutical product. It is important to provide accurate information to consumers of pharmaceutical products such that they can make informed decisions as to whether or not to purchase or consume a particular product. For instance, some consumers may have allergic reactions to certain pharmaceutical actives, and thus, cannot consume products containing such actives. In accordance with the methods provided herein, a packaged pharmaceutical product is provided. The product includes a complexate, as described above, an oral delivery system for delivery of the complexate and a package for containing the oral delivery system. Indicia identifying the complexate as an active ingredient contained in the packaged pharmaceutical product is provided. The indicia may be directly associated with the packaged pharmaceutical product. For example, the indicia may be a label on the product or an insert inside the product packaging. In addition, as an alternative to the presence of indicia on the package, indicia may also be placed on the consumable delivery system per se. In some other embodiments, the indicia may be indirectly associated with the product, such as a print, television or internet advertisement, or a product brochure at a physician's office. Consumers are exposed to the indicia, by reading the product packaging, viewing the advertisement, or the like, thereby providing accurate information as to the active ingredient contained in the packaged pharmaceutical product.

In some embodiments, the present invention also provides methods of marketing an oral delivery system containing a complexate as the active ingredient. In accordance therewith, a first pharmaceutical product is identified. This product contains an active that has been marketed to consumers as an effective pharmaceutical active. A second pharmaceutical product is provided, which includes a complexate formed from a complexing agent and the same active contained in the first pharmaceutical product. The product also includes an oral delivery system for delivery of the complexate. Consumers are educated that the complexate contained in the second pharmaceutical product provides the same effectiveness as the active contained in the first pharmaceutical product. Such education may be effected via advertisements, such as print, television and internet advertisements. Educational product information also may be supplied to physicians to disseminate to their patients. The second pharmaceutical product then may be marketed to consumers on this basis.

The present invention additionally is directed to methods of satisfying drug regulations promulgated by a regulatory body for an oral delivery system including a complexate as an active ingredient. In accordance with such methods, safety and efficacy test results for the complexing agent and safety and efficacy test results for the pharmaceutical active are submitted to the regulatory body, such as the FDA. A determination then is made as to whether or not additional safety and efficacy tests for the complexate are required by the regulatory body. If such additional tests are required to satisfy the regulations, these tests are conducted and the results are submitted to the regulatory body to satisfy the drug regulations for the oral delivery system. Additionally, a determination may be made as to whether or not a change in labeling on the package of the oral delivery system is required to satisfy drug regulations. In particular, the labeling may need to be changed to appropriately identify the complexate as an active ingredient contained in the product. If necessary, the product labeling also may be changed to satisfy the governmental drug regulations.

### EXAMPLES

### Example 1

Packaged pharmaceutical products of the present invention including oral delivery systems in the form of flash dose tablets for delivery of a complexate are prepared.

First, the complexate was prepared by placing 16.24 g of highly purified crosslinked polystyrene copolymer in sodium form (commercially available under the trade name Tulsion 344 from Thermax), which is the complexing agent, in an 8 ounce screw cap bottle with 125 ml of distilled water and stirring with a magnetic stirrer for 5 minutes. 5.41 g of loperamide HCl, which is the initial active, was added to the bottle and stirred with a magnetic stirrer for 16 hours with a screw cap on the bottle. The ratio of crosslinked polystyrene copolymer to loperamide HCl was 3:1. The reaction mixture was filtered through a Buchner funnel to separate the solid material from the liquid. The solid material was washed on the filter with distilled water. The filtrate was saved. The solid material collected on the filter was allowed to air dry.

The solid material then was dried in a 95° C. air oven in an aluminum foil dish for 2.5 hours to achieve a constant weight of dried material. 20.964 g of dried material was recovered. The dried solid material, which is the complexate, was sieved through a 60 mesh screen and stored in a screw cap bottle.

An oral delivery system in the form of a flash dose unit (tablet) incorporating the complexate is prepared as follows.

Initially, a shearform matrix material is prepared in accordance with the formula set forth in Table 1.

TABLE 1

| Component | Amount (wt. %) |
|---|---|
| Sugar (sucrose) | 84.75% |
| Binding agent (sorbitol) | 12.00% |
| Binding agent (α lactose) | 3.00% |
| Surfactant (Tween 80) | 0.25% |

The sucrose, sorbitol and lactose are mixed first by hand and then by machine until a homogenous blend is produced. To this mixture, the surfactant is added and mixed by hand. The blend is then subjected to flash flow processing in a Econo Floss Machine No. 7025 at approximately 3,600 rpm at a temperature setting of high. The spun material is collected as a floss and macerated in a mixing machine for about 45 seconds. The resulting material is a reduced volume shearform matrix in uncured condition.

A complexate mixture using the shearform matrix is prepared in accordance with the formula set forth below in Table 2.

TABLE 2

| Component | Amount (wt. %) |
|---|---|
| Shearform matrix (floss from Table 1) | 60.46% |
| Complexate | 32.09% |
| Flavor | 6.00% |
| High Intensity Artificial Sweetener | 0.80% |
| Lecithin | 0.35% |
| Silica | 0.25% |
| Orange color | 0.05% |

The lecithin and complexate are mixed and added to the ground floss material. The ingredients are mixed in a mechanical mixing apparatus for 15-20 seconds. The flavors, high intensity sweetener, silica are then added and mechanically mixed with an additional 10-15 seconds. Finally, the color is added and mixed until the blend takes on a homogenous orange color.

The ingredients mix well on a large scale. The mixture has a homogenous density and excellent flow characteristics. The mixture is added in portions of 0.75 grams to a die having a 0.65 inch diameter. The ingredients are then tamped at a pressure of 80 psi. The tamped dosage units are then cured.

The flash dose tablets prepared above include a complexate and can be packaged in accordance with the present invention. For example, the flash dose tablets are packaged in a blister pack. The blister pack includes a bottom layer with wells. Each well is adapted to contain a single tablet. The flash dose tablets are positioned within the wells of the blister pack, and a top layer is positioned over the bottom layer and sealed thereto. The outside of the blister pack, for example, the top layer, includes indicia listing the active ingredients contained in the flash dose tablets housed therein. The complexate contained in the tablets is included in this listing of active ingredients. The complexate itself is listed as a chemical entity in this listing of active ingredients, rather than the initial active, loperamide. Therefore, the package provides the consumer with appropriate information as to the active ingredients contained in the flash dose tablets.

### Example 2

Packaged pharmaceutical products of the present invention including oral delivery systems in the form of capsules for delivery of a complexate are prepared.

First, the complexate was prepared by placing 4 g of highly purified crosslinked polystyrene copolymer in sodium form (commercially available under the trade name Tulsion 344), which is the complexing agent, in a 50 ml beaker with 16 g of distilled water and stirring with a magnetic stirrer for 5 minutes. 2 g of dextromethorphan hydrobromide, which is the initial active, was added to the beaker and allowed to stir with the magnetic stirrer for about 3 hours and 45 minutes. The reaction mixture was allowed to settle and the water portion was decanted. Additional water was added to the beaker and allowed to settle. The water portion was again removed by decanting. The solid reaction product, which is the complexate, was dried for 15 hours in a 30° C. air oven.

An oral delivery system in the form of a soft gelatin capsule incorporating the complexate is prepared using the components listed in Tables 3 and 4.

TABLE 3
Liquid Core Composition

| Component | Amount (wt. %) |
|---|---|
| Complexate | 23.00 |
| Polyethylene glycol | 50.00 |
| Polyvinylpyrrolidone | 2.00 |
| Propylene glycol | 13.00 |
| Water | qs |

The complexate, polyethylene glycol, polyvinylpyrrolidone, propylene glycol and water are combined in a suitable vessel and warmed to 70° C. until a homogeneous solution is obtained.

TABLE 4
Gelatin Capsule

| Component | Amount (wt. %) |
|---|---|
| Gelatin | 47.00 |
| Glycerin | 15.00 |
| Water | qs |

The above ingredients are combined in a suitable vessel and heated with mixing at about 65° C. to form a uniform solution. Using standard encapsulation methodology, the resulting solution is used to prepare soft gelatin capsules containing the liquid core composition formed above. The resulting soft gelatin complexate capsules are suitable for oral administration.

The capsules prepared above include a complexate and can be packaged in accordance with the present invention. For example, a plurality of the capsules can be packaged in a bottle. The bottle has a label affixed to the external surface thereof. The label includes indicia listing the active ingredients contained in the capsules housed therein. The complexate contained in the capsules is included in this listing of active ingredients. The complexate itself is listed as a chemical entity in this listing of active ingredients, rather than the initial active, dextromethorphan hydrobromide. Therefore, the package provides the consumer with appropriate information as to the active ingredients contained in the capsules.

### Example 3

Packaged pharmaceutical products of the present invention including oral delivery systems in the form of coated caplets for delivery of a complexate are prepared.

First, the complexate was prepared by placing 13.5 g of highly purified crosslinked polystyrene copolymer in sodium form (commercially available under the trade name Tulsion 344), which is the complexing agent, in an 8 ounce screw cap bottle with 100 ml of distilled water and stirring with a magnetic stirrer for 5 minutes. 5.4 g of cetirizine dihydrochloride (commercially available as Zyrtec from Pfizer), which is the initial active, was added to the bottle and allowed to stir with the magnetic stirrer for 16 hours with a screw cap on the bottle. The ratio of the highly purified crosslinked polystyrene copolymer to cetirizine dihydrochloride was 2.5 to 1. The reaction mixture was filtered through a Buchner funnel to separate the solid material from the liquid. The solid material was washed on the filter with distilled water. The filtrate was saved.

The solid material collected on the filter was dried in a 95° C. air oven in an aluminum foil dish for 2 hours to achieve a constant weight of dried material. 17.06 g of dried material was recovered. The dried solid material, which is the complexate, was sieved through a 60 mesh screen and stored in a screw cap bottle.

An oral delivery system in the form of an uncoated caplet (capsule-shaped tablet) incorporating the complexate is prepared using the components listed in Table 5.

TABLE 5

| Component | Amount (mg) |
|---|---|
| Complexate | 500.00 |
| PVP | 5.00 |
| Corn starch | 50.55 |
| Stearic acid | 2.00 |

The components are blended and granulated, adding additional water as required. The wet mass is passed through a mill and dried in a fluid bed dryer. The dry granulation is passed though a mesh screen. The granulation is fed into a tablet die provided with a lower tablet punch. The granulations are then compressed by an upper tablet punch to form a tablet. The tablet die and tablet punches are tooled to form a capsule-shaped tablet, i.e., caplet.

The uncoated caplets are then film coated using the coating composition in Table 6.

The naproxen and cyclodextrin are added to water and stirred with a magnetic stirrer for 4 hours at 60° C. The solution is then evaporated using a rotary distillation unit under vacuum. The remaining powder is then dried to a constant weight using a mechanical oven at 80° C. to form the complexate.

An oral delivery system in the form of a controlled-release tablet incorporating the complexate is prepared using the components listed in Table 8.

TABLE 8

| Component | Amount (g/tablet) |
|---|---|
| Complexate | 550.0 |
| Hydroxypropylmethyl cellulose | 41.25 |
| Talc | 31.5 |
| Magnesium stearate | 3.5 |
| Deionized water | 36.0 |

The complexate and hydroxypropylmethyl cellulose are well blended and then granulated with the purified deionized water. The granulation is tray dried in a 50° C. oven for 12 hours, passed at slow speed through a hammer mill which is fitted with an 18 gauge screen, and then thoroughly mixed with the talc and magnesium stearate. The resulting homogenous matrix material is then pressed into tablets of uniform size and weight with 3500 pounds load compression.

The tablets include a complexate and can be packaged in accordance with the present invention. For example, the tablets can be packaged into blister packs, as described in Example 1, or in a bottle, as described in Example 2. Indicia are located on the outer surface of the blister pack or bottle, which lists the complexate as an active ingredient included in the tablets contained in the package. Therefore, the packaging appropriately indicates to consumers the active ingredients contained in the product.

TABLE 6

| Component | Amount (mg/caplet) |
|---|---|
| Uncoated caplet | 557.550 |
| PVP | 2.041 |
| Hydroxypropylmethyl cellulose | 10.883 |
| Propylene glycol | 1.496 |
| Arlacel - 20 | 0.812 |
| Tween - 20 | 0.612 |
| Mineral oil | 0.816 |
| Color agent | 0.068 |

The components listed in Table 6 are combined to form a film coating solution. The uncoated caplets are placed in a coating pan. The film coating solution is sprayed into the pan while drying. The caplets are cooled to room temperature in the coating pan with exhaust. The caplets then can be polished by sprinkling a polishing wax into the pan and mixing for about 5 minutes.

These coated caplets include a complexate and can be packaged in accordance with the present invention. For example, the coated caplets can be packaged into blister packs, as described in Example 1, or in a bottle, as described in Example 2. Indicia are located on the outer surface of the blister pack or bottle, which lists the complexate as an active ingredient included in the coated caplets contained in the package. Therefore, the packaging appropriately indicates to consumers the active ingredients contained in the product.

### Example 4

Packaged pharmaceutical products of the present invention including oral delivery systems in the form of tablets for delivery of a complexate are prepared.

First, the complexate is prepared including the components listed in Table 7.

TABLE 7

| Component | Amount (g) |
|---|---|
| Naproxen | 2.5 g |
| Cyclodextrin¹ | 10.0 g |
| Water | 100 g |

¹Cavamax W7, available from Wacker Chemie AG

What is claimed is:

1. A packaged pharmaceutical product comprising:
a complexate comprising a complexing agent and an active;
an oral delivery system for delivery of said complexate; and
a package for containing said oral delivery system, said package comprising indicia associated therewith identifying said complexate as a regulatory approvable chemical entity.

2. The pharmaceutical product of claim 1, wherein said active comprises a charged active.

3. The pharmaceutical product of claim 1, wherein said complexing agent comprises an anionic complexing agent.

4. The pharmaceutical product of claim 1, wherein said complexing agent comprises a cationic complexing agent.

5. The pharmaceutical product of claim 2, wherein said charged active comprises an anionic active.

6. The pharmaceutical product of claim 2, wherein said charged active comprises a cationic active.

7. The pharmaceutical product of claim 1, wherein said complexing agent comprises a multi-valent salt.

8. The pharmaceutical product of claim 1, wherein said complexing agent comprises a zeolite.

9. The pharmaceutical product of claim 1, wherein said complexing agent comprises an ion exchange resin.

10. The pharmaceutical product of claim 1, wherein said complexing agent comprises a caged molecule.

11. The pharmaceutical product of claim 10, wherein said caged molecule comprises a cyclodextrin.

12. The pharmaceutical product of claim 1, wherein said package is selected from the group consisting of: pouch, box, cassette, blister pack, bag, bottle, syringe, vial and tube.

13. The pharmaceutical product of claim 1, wherein said indicia comprises a label.

14. The pharmaceutical product of claim 13, wherein said label is located on said package.

15. The pharmaceutical product of claim 13, wherein said label is affixed to said package.

16. The pharmaceutical product of claim 1, wherein said indicia comprises a list of active ingredients.

17. The pharmaceutical product of claim 1, wherein said indicia identifies said complexate as an active ingredient.

18. A method of labeling a packaged pharmaceutical product in a manner that is consistent with governmental drug regulations, comprising the steps of:
(a) providing a packaged pharmaceutical product comprising:
(i) a complexate comprising a complexing agent and a pharmaceutical active;
(ii) an oral delivery system for delivery of said complexate; and
(iii) a package for containing said oral delivery system;
(b) adding indicia to said package, said indicia comprising a list of active ingredients contained in said oral delivery system and a list of inactive ingredients contained in said oral delivery system, wherein said complexate is listed as an active ingredient.

19. The method of claim 18, wherein said indicia added to said package in step (b) identifies said complexate as a regulatory approvable chemical entity.

20. A method of adjusting the sales price of a packaged oral delivery system, comprising the steps of:
(a) selecting an oral delivery system comprising a pharmaceutical active, said product having a manufacturing cost;
(b) selecting a complexing agent for use with the pharmaceutical active to form a complexate;
(c) estimating the cost of purchasing the complexing agent from a supplier;
(d) estimating the cost of manufacturing the complexate;
(e) determining the change in manufacturing cost of the oral delivery system based on the additional cost estimates of steps (c) and (d); and
(f) adjusting the sales price of the product based on the change in manufacturing cost.

21. The method of claim 20, further comprising the steps of:
(g) determining whether additional safety and efficacy tests associated with the complexate are required; and
(h) estimating the cost associated with the additional safety and efficacy tests,
wherein step (e) further comprises determining the change in manufacturing cost of the oral delivery system based on the additional cost estimate of step (h).

22. The method of claim 20, further comprising the steps of:
(i) determining whether a change in labeling on the product package is required to list the complexate as an active ingredient; and
(j) estimating the cost associated with the labeling change,
wherein step (e) further comprises determining the change in manufacturing cost of the oral delivery system based on the additional cost estimate of step 0).

23. The method of claim 20, further comprising the step of determining whether the adjusted sales price of step (f) is viable in the marketplace.

24. A method of disseminating accurate drug information to consumers of a packaged pharmaceutical product, comprising the steps of:
(a) providing a packaged pharmaceutical product comprising:
(i) a complexate comprising a complexing agent and a pharmaceutical active;
(ii) an oral delivery system for delivery of said complexate; and
(iii) a package for containing said oral delivery system;
(b) providing indicia identifying the complexate as an active ingredient contained in the packaged pharmaceutical product; and
(c) exposing consumers to the indicia, thereby providing accurate information as to the active ingredient contained in the packaged pharmaceutical product.

25. A method of marketing an oral delivery system containing a complexate as the active ingredient to consumers, comprising the steps of:
(a) identifying a first pharmaceutical product containing an active that has been marketed to consumers as an effective pharmaceutical product;
(b) providing a second pharmaceutical product comprising:
(i) a complexate comprising a complexing agent and the active of step (a); and
(ii) an oral delivery system for delivery of the complexate;
(c) educating consumers that the complexate contained in the second pharmaceutical product provides the same effectiveness as the active contained in the first pharmaceutical product; and
(d) marketing the second pharmaceutical product to consumers.

26. A method of developing a sales price for a packaged oral delivery system including a complexate as the active ingredient, comprising the steps of:
(a) selecting a pharmaceutical active and complexing agent to form the complexate included in the oral delivery system;
(b) estimating the cost of purchasing the complexing agent from a supplier;
(c) estimating the cost of purchasing the pharmaceutical active from a supplier;
(d) estimating the cost of manufacturing the complexate from the pharmaceutical active and the complexing agent;
(e) estimating the cost of producing and packaging the oral delivery system including the complexate;
(f) estimating the aggregate cost of safety and efficacy tests performed for the pharmaceutical active and the complexing agent individually;
(g) determining whether additional safety and efficacy tests for the complexate are required;
(h) estimating the cost associated with the additional safety and efficacy tests of step (g);
(i) adding the costs of the preceding steps to develop an estimated total manufacturing cost; and
(j) setting the sales price based on the estimated total manufacturing cost of the packaged oral delivery system.

27. A method of satisfying drug regulations promulgated by a regulatory body for an oral delivery system comprising a complexate as the active ingredient, said complexate comprising a complexing agent and a pharmaceutical active, comprising the steps of:
(a) submitting safety and efficacy test results for the complexing agent and safety and efficacy test results for the pharmaceutical active to the regulatory body;
(b) determining whether additional safety and efficacy tests for the complexate are required by the regulatory body;
(c) conducting the safety and efficacy tests of step (b); and
(d) submitting the results of the safety and efficacy tests of step (c) to the regulatory body to satisfy the drug regulations of the regulatory body for the oral delivery system.

28. The method of claim 27, further comprising the step of determining whether a change in labeling on the package for the oral delivery system is required by the regulatory body to list the complexate as an active ingredient.
